(19) United States
(12) Patent Application Publication
(10) Pub. No.: US 2006/0200187 A1
(43) Pub. Date: Sep. 7, 2006

(54) Childbirth inflatable water/air vaginal obturator

(76) Inventor: Charlie F. Gude, Rockford, IL (US)

Correspondence Address: Donald W. Meeker;Patent Agent, 924 East Ocean Front #E, Newport Beach, CA, 92661, US

(21) Appl. No.: 11/071,608

(22) Filed: Mar. 3, 2005

(51) Int. Cl. A61M 29/00

(52) U.S. Cl. 606/193

(57) ABSTRACT

A hollow shaft has a Y-shaped handle at one end and an inflatable bladder at the other end. The bladder is shaped and sized to simulate a presenting part of a fetus at birth based on ultrasound or other fetal examination. Air or water or both inflate the bladder using a pump controlled by buttons and a pressure gauge adjacent to the handle. The bladder stretches vaginal tissue and surrounding tissue to the size and shape of the presenting parts of the fetus during birth.

### BACKGROUND OF THE INVENTION

1. Field of the Invention

The present invention relates to vaginal dilators and particularly to a vaginal obturator which comprises a tube with a Y-shaped handle at one end and an air or water or combined air-water receiving inflatable bladder at the other end for expanding the vaginal tissue and surrounding tissue prior to the delivery of a baby, the inflatable bladder shaped and sized to simulate the presenting parts of the fetus during childbirth as determined by ultrasound or other fetal examination.

2. Description of the Prior Art

Current obstetrical practice is to prevent tearing of the muscles of the vagina and perineum during childbirth by performing an episiotomy to create a regular laceration, which is later sutured. However, both natural tearing of the vaginal and perineal muscles during delivery and surgical episiotomies are subject to further complications. The use of a vaginal obturator prior to childbirth will reduce the incidence of lacerations of the muscles of the vagina and perineum. Expanding the vaginal opening eliminates many of the complications of childbirth and can shorten or avoid prolonged labor.

Prior art devices are not always easy to use or easy to control and don't always provide a dilating device of the precise size and shape of the presenting portion of the fetus.

U.S. Patent Application #20030087734, published May 8, 2003 by Kring, discloses a vaginal-pelvic muscle exerciser and birthing trainer. A device is provided for insertion into the birth canal of a female user, which comprises an asymmetrical, inflatable, bulb defining an outer surface, the bulb sized and shaped so as to define a crease along at least a portion of the surface adapted to be placed in registry with the user's urethra when the bulb is inflated, the bulb further sized and shaped to contact the bulbocavernous facia, pubococcygeal facia and illiococcygeal facia when inflated, and means for inflating and deflating the bulb.

U.S. Patent Application #20020010441, published Jan. 24, 2002 by Horkel, describes a device for preparing a pregnant woman for delivery and facilitating the delivery itself. Said device comprises an entirely expandable stretch body, which is positioned in the expulsion area of the urogenital canal in such a way that it is partly inside the vagina and partly outside. The invention also relates to preparatory gymnastics method involving the device.

U.S. Pat. No. 4,137,922, issued Feb. 6, 1979 to Leininger, discloses a novel inflatable device for use in dilating various body cavities, and especially the human cervix. The device comprises an envelope member having an enlarged bulbous portion on one end, and a shield at the opposite end. The envelope member is inflatable with suitable gasses such as carbon dioxide, or liquids such as saline, distilled water and the like.

U.S. Pat. No. 3,626,949, issued Dec. 14, 1971 to Shute, concerns a cervical dilator, which comprises a PVC envelope or case having a hose connection for filling up the case with a fluid under pressure. The case can be inserted into the cervical canal while folded up and is then periodically expanded under pressure such that the case expands radially against the cervical wall to thereby simulate contractions. Between opposed ends of the case there is a waist portion which is not expandable in a radial direction, which waist portion is positioned inside the edge of the cervix in order to prevent that the expanded waist portion moves in an axial direction under pressure.

U.S. Pat. No. 3,480,017, issued Nov. 25, 1969 to Shute, illustrates a cervical dilator that uses an inflatable case, which is inflatable to a substantially disk-shaped body, which is positioned in use inside the uterus and inflatable using an inflation tube whose opening is inside the case. The case comprises a circumferential constriction such that a groove is formed in the cylindrical exterior wall of the case when inflated into a disk. The cervix supported by the groove can be expanded in this way in order to induce birth. The application of the known device is only possible by a doctor who has acquired the skills to manipulate the shell against the cervix using the inflating tube as a handle, without damaging the amniotic sack or the fetus' head.

U.S. Pat. No. 5,865,729, issued Feb. 2, 1999 to Meehan, shows a vaginal speculum having an inflatable bladder, which includes a central channel and an inlet outlet port. The inflatable bladder may include optical fibers for illumination.

U.S. Pat. No. 6,224,580, issued May 1, 2001 to Christensen, illustrates an apparatus for use in the antenatal period of pregnant women for insertion into the vagina, the main function of which is to dilate muscular, tendinous and connective tissue in the pelvic floor so as to make these tissue structures more flexible. The apparatus includes a length of hose mounted on a mandrel and is expandable by means of a fluid, the length of hose being connectable with the fluid through at least one channel in the mandrel. The mandrel is formed with at least one channel, which extends longitudinally through it and terminates outside the user in the use of the apparatus. The mandrel with the length of hose may be provided with a replaceable cover, which adds to the hygiene and safety.

U.S. Pat. No. 899,477, issued Sep. 22, 1908 to Williams, puts forth a dilator and applicator for the treatment of the vagina, rectum and other body cavities. The device comprises: a casing with an inlet and an outlet, which are connected to a liquid source and a waste receptacle, respectively by means of flexible ducts or tubes; a bag made of distensible material with an open end, which is fitted to an end of the casing and secured detachably by means of a band or a clasp; and an ordinary syringe bulb, which is used to force liquid through a hose into the inlet of the casing.

One of the US patents refers to a French patent as follows: In French Patent publication FR-592,104, there is described a dilator to be inserted in the perineum which is made of an elongated elastic material balloon which can be laterally expanded by inflation while having its lengthwise expansion limited. By methodical use and progressive expansion, tissue having undergone such exercise will yield while giving birth without tearing.

What is needed is an easy-to-grip and easy-to-control hand held instrument with a valve to insufflate and deflate various sizes and shapes of inflatable bladders inside the vagina to stretch the vaginal tissue and surrounding tissue to the precise size and shape of the presenting parts of the fetus about to pass through during birth, the inflatable bladder sized to simulate the head and/or other presenting parts of the fetus which will pass through the birth canal and vagina at birth based on estimations and previous ultrasound or other fetal measurement that would have been done prior to birth showing various head circumferences and orientations of the fetus in the womb just prior to delivery.

### SUMMARY OF THE INVENTION

An object of the present invention is to provide an easy-to-grip and easy-to-control hand held instrument with a valve to insufflate and deflate various sizes and shapes of inflatable bladders inside the vagina to stretch the vaginal tissue and surrounding tissue to the size of the presenting parts of the fetus about to pass through during birth, the balloon sized to simulate the head and/or other presenting parts of the fetus which will pass through the birth canal and vagina at birth based on estimations and previous ultrasound or other fetal examination that would have been done prior to birth showing various head circumferences and orientations of the fetus in the womb just prior to delivery to decrease the need for tearing of the vagina or episiotomy during childbirth and allow the mother to deliver the baby comfortably with fewer complications and allow the labor to progress sooner without the need for excessive drugs to stimulate the uterus to contract resulting in less pain for the mother and less drugs needed in delivery and avoid or shorten traumatic prolonged labor.

In brief, an easy-to-grip and easy-to-control hand held instrument with a valve to insufflate and deflate a precisely sized and shaped inflatable bladder inside the vagina to stretch the vaginal tissue and surrounding tissue to the size of the presenting parts of the fetus about to pass through during birth. The inflatable bladder is sized and shaped to simulate the head and/or other presenting parts of the fetus which will pass through the birth canal and vagina at birth based on estimations and previous ultrasound or other fetal examinations that would have been done prior to birth showing various head circumferences and orientations of the fetus in the womb just prior to delivery.

Because the inflatable bladders will be made to order it will assure a good fit, form and function in assisting the mother in delivery. It also will not require sterilization since it technically is not a surgery therefore any physician or midwife able to deliver a baby would find it of great assistance.

An advantage of the present invention is that it provides an easy-to-grip and easy-to-control hand held instrument to stretch the vaginal tissue and surrounding tissue to the size of the presenting parts of the fetus about to pass through during birth. An additional advantage of the present invention is that the inflatable bladders are sized to simulate the head and/or other presenting parts of the fetus which will pass through the birth canal and vagina at birth.

Another advantage of the present invention is that it decreases the need for tearing of the vagina or need for episiotomy of the vagina and the surrounding tissues during childbirth.

Still another advantage of the present invention is that it allows the mother to deliver the baby safely and comfortably with fewer complications.

Yet another advantage of the present invention is that it allows the labor to progress sooner without the need for excessive drugs to stimulate the uterus to contract resulting in less pain for the mother and less drugs needed in delivery.

One more advantage of the present invention is that it provides a way to avoid or shorten traumatic prolonged labor.

Still one more advantage of the present invention is that the balloons will be made to order assuring good fit, form and function in assisting the mother in delivery.

Yet one more advantage of the present invention is that it will not require sterilization since it technically is not a surgery therefore any physician or midwife able to deliver a baby would find it of great assistance.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a diagrammatic plan view of the vaginal obturator of the present invention;

FIG. 1A is a diagrammatic elevational view in partial section between the two arms of the Y-shaped handle of the vaginal obturator taken through A-A of FIG. 1 showing two inlet ports, one for air and one for water;

FIG. 2 is a diagrammatic plan view of an inflatable bladder of the vaginal obturator of the present invention having a configuration and size of a head and shoulder of a fetus just prior to childbirth;

FIG. 3 is a diagrammatic plan view of an inflatable bladder of the vaginal obturator of the present invention having a configuration and size of a head of a fetus just prior to childbirth;

FIG. 4 is a diagrammatic plan view of an inflatable bladder of the vaginal obturator of the present invention having a configuration and size of a head of a fetus just prior to childbirth with a water/air interface shown inside the inflatable bladder.

### BEST MODE FOR CARRYING OUT THE INVENTION

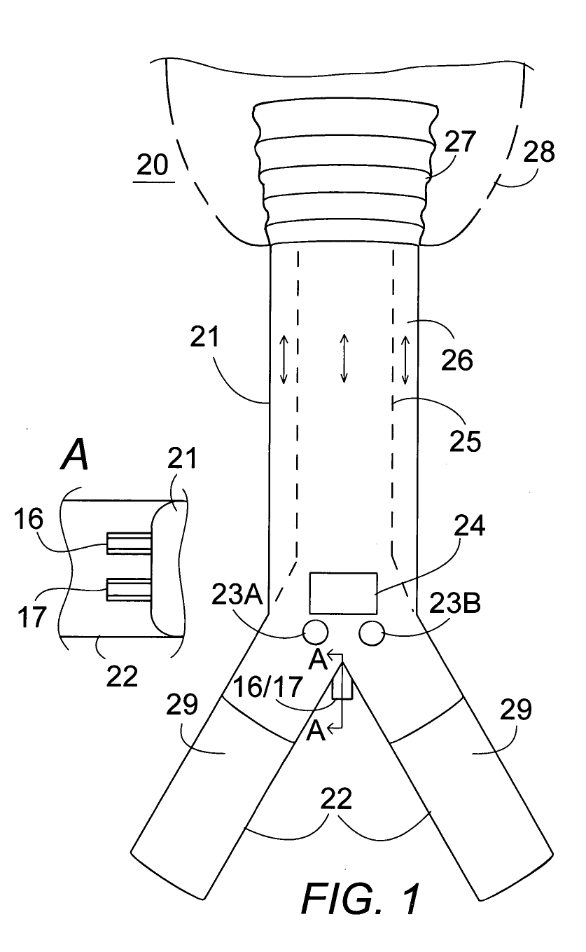

In FIGS. 1 through 4, a vaginal obturator device to prepare a vaginal area for delivery of a fetus during childbirth comprises a vaginal obturator instrument 20, which comprises an elongated hollow shaft 21 insertable in a vaginal opening during childbirth, a smoothly grooved distal tip of the shaft 27 structured to receive and retain any of a variety of sizes and shapes of inflatable bladders 28, 28A, 28B, and 28C for expanding inside a vagina just prior to childbirth to expand the vaginal tissue and surrounding tissue to a size of a head of a fetus about to be born.

The inflatable bladder is sized to simulate the head and/or other presenting parts of the fetus which will pass through the birth canal and vagina at birth based on estimations and ultrasound measurement made prior to birth showing various head circumferences and orientations of the fetus in the womb just prior to delivery to decrease the need for tearing of the vagina or need for episiotomy (cutting) of the vagina and the surrounding tissues during childbirth and allow the mother to deliver the baby comfortably with fewer complications and allow the labor to progress sooner without the need for excessive drugs to stimulate the uterus to contract resulting in less pain for the mother and less drugs needed in delivery.

A pump means (not shown) in communication with the inflatable bladder may be an external pump or a miniature pump inside the vaginal obturator instrument for pumping at least one fluid into the inflatable bladder to expand the inflatable bladder to stretch the vaginal tissue and surrounding tissue and alternately pumping the fluid out of the inflatable bladder to deflate the bladder in order to remove the vaginal obturator instrument from the vaginal opening.

A Y-shaped handle 22 with hand grips 29 over each arm of the Y-shaped handle 22 is attached to a proximal end of the shaft for grasping the vaginal obturator instrument with two hands, one hand on each side of the Y-shaped handle, to insert the shaft 21 in the vaginal opening and withdraw the shaft from the vaginal opening. A means for controlling the pump means 23A and 23B is positioned externally on the vaginal obturator instrument immediately adjacent to the Y-shaped handle 22 for easy access and use. The control means comprising two buttons, one button 23B for insufflating the inflatable bladder and another button 23A for deflating the inflatable bladder.

A gauge 24 is positioned exteriorly on the vaginal obturator instrument adjacent to the Y-shaped handle 22 in a location viewable by a person grasping the Y-shaped handle 22. The gauge indicates pressure inside the inflatable bladder to allow for safe accurate inflation.

The vaginal obturator instrument may further comprise an inner chamber 25 within the hollow shaft 21. The inner chamber 25 having an inner chamber interior passageway, shown by the center double arrow in FIG. 1, communicating with the inflatable bladder 28. The inner chamber 25 is spaced apart from the outer casing of the hollow shaft 21 to form an annular interior passageway 26 between the inner chamber and the outer wall, shown by the two side double arrows in FIG. 1, the annular passageway communicating with the inflatable bladder 28.

In FIG. 1 and FIG. 1A, the vaginal obturator instrument 20 further comprises an air inlet 16 into the vaginal obturator instrument communicating with one of the interior passageways and a water inlet 17 into the vaginal obturator instrument communicating with the other of the interior passageways. The fluid in vaginal obturator instrument may be air or water or both through hoses (not shown) attached to the two inlets 16 and 17.

The hand grips 29 on the vaginal obturator instrument may be custom plastic grips or custom rubber grips.

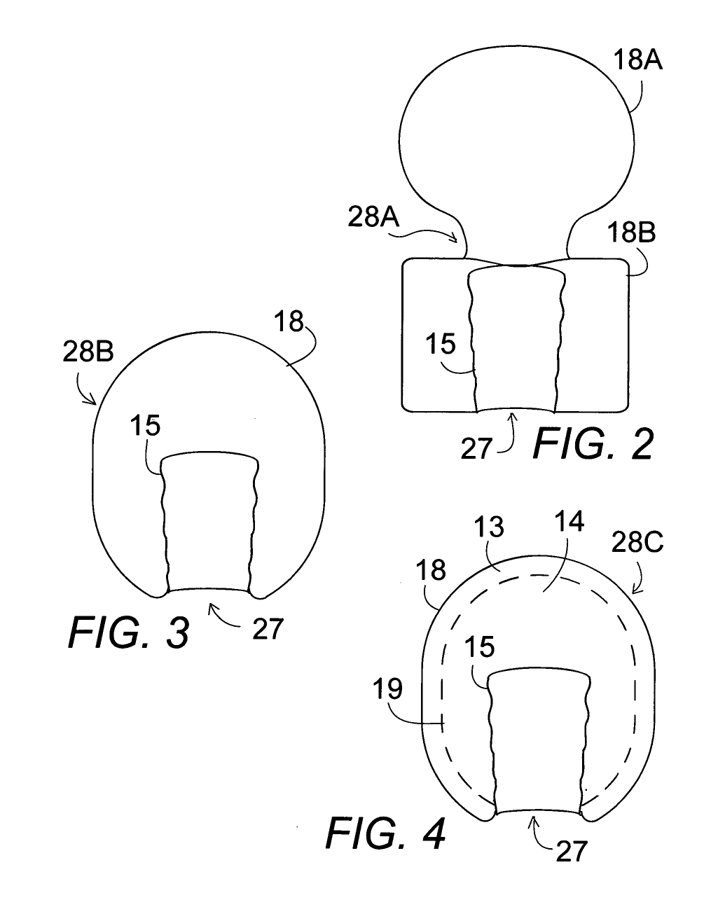

In FIG. 2, the inflatable bladder 28A is structured to simulate the size and shape of the fetus' head and shoulders with a head-shaped portion 1 8A of the bladder and shoulder shaped portion 18B of the bladder.

In FIG. 3, the inflatable bladder 28A is structured to simulate the size and shape of the fetus' head with a head-shaped portion 18 of the bladder.

In FIG. 4, the inflatable bladder is structured to simulate the size and shape of the fetus' head with a head-shaped portion 18 of the bladder. The inflatable bladder further comprises an inner inflatable bladder 19 within the outer head shaped portion 18 of the bladder, the inner inflatable bladder 19 is spaced apart from the outer head shaped portion 18 of the bladder to form an exterior chamber 13 between the inner bladder 19 of the bladder and the outer head shaped portion 18 of the bladder, and an interior chamber 14 inside the inner inflatable bladder 14. One chamber contains air and the other chamber contains water.

In FIGS. 2-4, an interior tight fitting annular connecting portion 15 of the bladder is adhered to the smoothly grooved distal tip of the shaft 27 which is inserted therein, as indicated by the bottom arrow.

In use, an ultrasound or other examination of the fetus determines the which parts of the fetus will present themselves first during childbirth and the sizes and shapes of the presenting parts. The precisely sized and shaped inflatable bladder 28, 28A, 28B, or 28C is adhered to the distal tip 27 of the vaginal obturator instrument 20.

The vaginal obturator instrument 20 is grasped with two hands on the Y-shaped handle 22 and the hollow shaft 21 inserted in the vagina just prior to childbirth. The inflator button 23B is pressed to inflate the inflatable bladder 28, 28A, 28B, or 28C to the pressure level indicated on the pressure gauge 24 which shows that the inflatable bladder is inflated to the desired pressure to expand the vaginal tissue and surrounding tissue to the size and shape of the presenting parts of the fetus. The deflator button 23A is then pressed to deflate the inflatable bladder and the vaginal obturator instrument 20 is removed from the vagina to allow the fetus to pass through.

The inflatable bladder is fabricated of a heavy weight latex or other highly expansible material to allow for inflation and deflation.

It is understood that the preceding description is given merely by way of illustration and not in limitation of the invention and that various modifications may be made thereto without departing from the spirit of the invention as claimed.

What is claimed is:

1. A vaginal obturator device to prepare a vaginal area for delivery of a fetus during childbirth, the device comprising:
a vaginal obturator instrument comprising an elongated hollow shaft insertable in a vaginal opening during childbirth, a smoothly grooved distal tip of the shaft, an inflatable bladder secured to the distal tip of the shaft, the inflatable bladder sized to simulate the head and/or other presenting parts of the fetus which will pass through the birth canal and vagina at birth based on fetal examination, a pump means in communication with the inflatable bladder for pumping at least one fluid into the inflatable bladder to expand the inflatable bladder to stretch the vaginal tissue and surrounding tissue and alternately pumping the at least one fluid out of the inflatable bladder to deflate the bladder in order to remove the vaginal obturator instrument from the vaginal opening to admit a fetus therethrough, a Y-shaped handle with hand grips over each arm of the Y-shaped handle attached to a proximal end of the shaft for grasping the vaginal obturator instrument with two hands, one hand on each side of the Y-shaped handle, to insert the shaft in the vaginal opening and withdraw the shaft from the vaginal opening, a means for controlling the pump means positioned externally on the vaginal obturator instrument immediately adjacent to the Y-shaped handle, the control means comprising two buttons, one for inflating the inflatable bladder and one for deflating the inflatable bladder, a gauge positioned exteriorly on the instrument adjacent to the Y-shaped handle in a location viewable by a person grasping the Y-shaped handle, the gauge indicating pressure inside the inflatable bladder.

2. The device of claim 1 wherein the fetal examination comprises an ultrasound examination.

3. The device of claim 1 wherein the smoothly grooved distal tip of the shaft is structured to receive and retain of a variety of sizes and shapes of inflatable bladders.

4. The device of claim 1 further comprising an inner chamber within the hollow shaft, the inner chamber having an inner chamber interior passageway communicating with the inflatable bladder, the inner chamber spaced apart from an outer wall of the hollow shaft to form an annular interior passageway between the inner chamber and the outer wall, the annular passageway communicating with the inflatable bladder.

5. The device of claim 4 further comprising an air inlet into the vaginal obturator instrument communicating with one of the interior passageways and a water inlet into the vaginal obturator instrument communicating with the other of the interior passageways.

6. The device of claim 1 wherein the fluid is air.

7. The device of claim 1 wherein the fluid is water.

8. The device of claim 1 wherein the fluid is air and water.

9. The device of claim 1 wherein the hollow shaft is nine inches in length.

10. The device of claim 1 further comprising a custom plastic grip over each of two arms of the Y-shaped handle.

11. The device of claim 1 further comprising a custom rubber grip over each of the two arms of the Y-shaped handle.

12. The device of claim 1 wherein the pump means is external to the vaginal obturator instrument.

13. The device of claim 1 wherein the pump means is positioned internally in the vaginal obturator instrument.

14. The device of claim 1 wherein the inflatable bladder comprises an inner bladder having an inner chamber therein, the inner bladder spaced apart from an outer bladder to form an outer chamber therebetween, the fluid in one of the chambers comprising air and the fluid in the other of the chambers comprising water.
